(19) United States
(12) Patent Application Publication
(10) Pub. No.: US 2021/0312296 A1
(43) Pub. Date: Oct. 7, 2021

(54) CLASSIFICATION OF SUBJECT-INDEPENDENT EMOTION FACTORS

(71) Applicant: Hewlett-Packard Development Company, L.P., Spring, TX (US)

(72) Inventors: Zhiyuan Li, Palo Alto, CA (US); Jishang Wei, Guilford, CT (US); Rafael Ballagas, Palo Alto, CA (US)

(73) Assignee: Hewlett-Packard Development Company, L.P., Spring, TX (US)

(21) Appl. No.: 17/259,966

(22) PCT Filed: Nov. 9, 2018
(86) PCT No.: PCT/US2018/060162

§ 371 Date: Jan. 13, 2021

(51) Int. Cl. G06N 3/08; G06N 3/04; A61B 5/16; A61B 5/00

(52) U.S. Cl. CPC G06N 3/088; A61B 5/7267; A61B 5/165; G06N 3/0454

(57) ABSTRACT

A method of removing individual variation from emotional representations may include classifying physiological data based on subject-independent emotion factors. The subject-independent emotion factors are isolated from subject-dependent individual factors. Further, a non-transitory computer readable medium includes computer usable program code embodied therewith. The computer usable program code, when executed by the processor classifies, with a first neural network, the physiological data based on subject-independent emotion factors from the trained first neural network. The subject-independent emotion factors have been isolated within the physiological data from subject-dependent individual factors.

### BACKGROUND

Enhanced reality systems and devices may be used to present to a user a virtual or augmented reality. This enhanced reality environment may be used for entertainment and professional purposes. Feedback may be provided to a user of an enhanced reality system or device in order to give the user the sensation of an immersion within the enhanced reality environment.

### BRIEF DESCRIPTION OF THE DRAWINGS

The accompanying drawings illustrate various examples of the principles described herein and are part of the specification. The illustrated examples are given merely for illustration, and do not limit the scope of the claims.

FIG. 1 is a block diagram of a system for classifying physiological data immune to individual variations, according to an example of the principles described herein.

FIG. 2 is a block diagram of a system for classifying physiological data immune to individual variations, according to an example of the principles described herein.

FIG. 3 is a block diagram of an unsupervised neural network, according to an example of the principles described herein.

FIG. 4 is a block diagram of a supervised neural network, according to an example of the principles described herein.

FIG. 5 is a block diagram of an encoder, according to an example of the principles described herein.

FIG. 6 is a flowchart showing a method of removing individual variation from emotional representations, according to an example of the principles described herein.

FIG. 7 is a flowchart showing a method of removing individual variation from emotional representations, according to an example of the principles described herein.

FIG. 8 is a flowchart showing a method of removing individual variation from emotional representations, according to an example of the principles described herein.

FIG. 9 is a flowchart showing a method of removing individual variation from emotional representations, according to an example of the principles described herein.

Throughout the drawings, identical reference numbers designate similar, but not necessarily identical, elements. The figures are not necessarily to scale, and the size of some parts may be exaggerated to more clearly illustrate the example shown. Moreover, the drawings provide examples and/or implementations consistent with the description; however, the description is not limited to the examples and/or implementations provided in the drawings.

### DETAILED DESCRIPTION

The ability to detect an individual's physiological state is helpful in providing feedback to that individual. For example, an individual's emotional state, cognitive load, stress, and even an onset of motion sickness, among a myriad of other physiological states may be diagnosed and action regarding that physiological state may be taken by a medical practitioner or may be performed autonomously by a computing device such as an enhanced reality system.

In order to better understand an individual's physiological state and provide feedback in the form of a diagnosis to the individual or an adjustment in a system such as a computer system, the individual's physiological state may be classified using a neural network. A neural network may be any artificial neural network, sometimes referred to as neuromorphic and synaptronic computation systems, that are computational systems that permit electronic systems to function in a manner analogous to that of biological brains. A neural network does not utilize a digital model of manipulating 0s and 1s, but, instead, creates connections between processing elements that are roughly functionally equivalent to neurons of a biological brain. A neural network may include various electronic circuits that are modeled on biological neurons. In a neural network, hardware and software serve as neurons that have a given activation function that operates on the inputs. By determining proper connection weights (a process also referred to as “training”), a neural network achieves efficient recognition of desired patterns, such as images and characters within a set of input data. In an example, these neurons may be grouped into “layers” in order to make connections between groups more obvious and to each computation of values. Training the neural network is a computationally intensive process.

Classification of an individual's physiological state may be performed using the neural network, and feedback may be provided. However, because elements of physiological data may vary from one individual to another, the neural network may find it difficult to classify the physiological states of several different individuals. For example, the physiological data may be obtained from an individual through a number of electrode or other sensors placed on the individual's body, and a number of signals may be produced that indicate a physiological state of the individual during the testing period. Within these signals, both subject-independent physiological factors and subject-dependent individual factors exist. In this situation, the inclusion of subject-dependent individual factors within the signal may bias a subsequent classification of the physiological data, whereas if the subject-independent physiological factors alone were used to classify the physiological data without the subject-dependent individual factors, the classification of the physiological data may be more precise and accurate.

In one example, the physiological signals may be provided to the neural network that is to be trained and used to classify the physiological data as it is received from an enhanced reality system. An enhanced reality system may be used to capture the physiological data, and the classified physiological data provided via the neural network may be the basis on which feedback to the individual is provided via the enhanced reality system. Although an enhanced reality system is described herein as the example system that provides physiological data to the neural network and the system that may provide the feedback based on the classified physiological data.

Examples described herein provide a method of removing individual variation from emotional representations. The method may include classifying physiological data based on subject-independent emotion factors. The subject-independent emotion factors are isolated from subject-dependent individual factors.

The method may include separating, with a neural network, the subject-dependent individual factors of physiological data from subject-independent emotion factors of the physiological data. This may include, with an individual-disentanglement encoder, separating the subject-dependent individual factors from a physiological data signal pair to create a first individual latent vector and a second individual latent vector. The separation of the subject-dependent individual factors from the physiological data signal pair creates a learned individual-disentanglement encoder. Further, the method may include, with an emotion-disentanglement encoder, separating the subject-independent emotion factors from the physiological data signal pair to create a first emotion latent vector and a second emotion latent vector. The separation of the subject-independent emotion factors from the physiological data signal pair creates a learned emotion-disentanglement encoder. As used in the present specification and in the appended claims, the terms “learned” and “trained” are used exchangeably and are meant to be understood broadly as any instance in which a computer generates rules (i.e., machine learning) underlying or based on raw data that has been fed into it using statistical techniques considered within the field of artificial intelligence. The statistical techniques overcome following strictly static program instructions by making data-driven predictions or decisions through building a model from sample inputs. Learning or training may include any form of supervised or unsupervised machine learning.

The method may also include applying a contrastive loss function to the first and second individual latent vectors, and applying the contrastive loss function to the first and second emotion latent vectors. The contrastive loss function causes the individual-disentanglement encoder and the emotion-disentanglement encoder to map the physiological data signal pair as close as possible in individual or emotion latent space if the physiological data signal pair are from the same individual or share the same emotion respectively. The method also include, with a decoder, reconstructing corresponding individual and emotion latent vectors to output reconstructed data. The final loss of this auto-encoder is a weighted sum of the contrastive loss and the reconstruction loss, and stochastic gradient descent is applied to updating weights in training process. Classifying the physiological data based on the subject-independent emotion factors may include, firstly, for each physiological signal of the physiological data signal, applying the learned emotion-disentanglement encoder to disentangle the subject-independent emotion factors from each physiological data signal, and secondly classifying the subject-independent emotion factors.

Examples described herein also provide a non-transitory computer readable medium includes computer usable program code embodied therewith. The computer usable program code, when executed by the processor, classifies, with a first neural network, the physiological data based on subject-independent emotion factors from the trained first neural network. The subject-independent emotion factors have been isolated within the physiological data from subject-dependent individual factors.

The computer readable medium may include computer usable program code to, when executed by the processor, train a second neural network to isolate subject-dependent individual factors of physiological data from subject-independent emotion factors of the physiological data, and with an individual-disentanglement encoder of the first neural network, separate the subject-dependent individual factors from a physiological data signal pair to create a first individual latent vector and a second individual latent vector. The separation of the subject-dependent individual factors from a physiological data signal pair creates a learned individual-disentanglement encoder. Further, computer readable medium may include computer usable program code to, when executed by the processor, separate, with an emotion-disentanglement encoder, the subject-independent emotion factors from the physiological data signal pair to create a first emotion latent vector and a second emotion latent vector. The separation of the subject-independent emotion factors from the physiological data signal pair creates a learned emotion-disentanglement encoder.

The computer readable medium may further include computer usable program code to, when executed by the processor, apply a contrastive loss function to the first and second individual latent vectors, apply the contrastive loss function to the first and second emotion latent vectors, and with a decoder, reconstruct the corresponding individual latent vectors and emotion latent vectors to output reconstructed data, wherein the contrastive loss function causes the individual-disentanglement encoder and the emotion-disentanglement encoder to map the physiological data signal pair in individual or emotion latent space as close as possible if the physiological data signal pair are from the same individual or share the same emotion respectively. Classifying the physiological data based on the subject-independent emotion factors includes, for each physiological signal, applying the learned emotion-disentanglement encoder to classify the subject-independent emotion factors from each physiological data signal, and classifying the subject-independent emotion factors.

The computer readable medium may also include computer usable program code to, when executed by the processor, and for each of the individual-disentanglement encoder and the emotion-disentanglement encoder, apply a plurality of convolutional layers of a parallel convolutional neural network (CNN) to a corresponding number of bands of the physiological data, and concatenate features learned from channels of the parallel CNN to produce the first individual latent vector, the second individual latent vector, the first emotion latent vector, and the second emotion latent vector, respectively. Weighting factors applied to each of the of the of convolutional layers are not the same.

Examples described herein also provide a system for classifying physiological data immune to individual variations. The system may include a supervised neural network to classify the physiological data based on subject-independent emotion factors. The subject-independent emotion factors have been isolated within the physiological data from subject-dependent individual factors.

The system may include an unsupervised neural network to isolate the subject-dependent individual factors of physiological data from the subject-independent emotion factors of the physiological data. The unsupervised neural network may include an individual-disentanglement encoder to separate the subject-dependent individual factors from the physiological data to create a first individual latent vector and a second individual latent vector, an emotion-disentanglement encoder to separate the subject-independent emotion factors from the physiological data to create a first emotion latent vector and a second emotion latent vector, a first contrastive loss module to apply a contrastive loss function to the first and second individual latent vectors, a second contrastive loss module to apply the contrastive loss function to the first and second emotion latent vectors, and a decoder to reconstruct the corresponding individual latent vector and emotion latent vector to output reconstructed data. The learnt second variational encoder is applied to the supervised neural network as a machine learning process. The system may be an enhanced reality system, and the physiological data is obtained from a peripheral augmented reality input device.

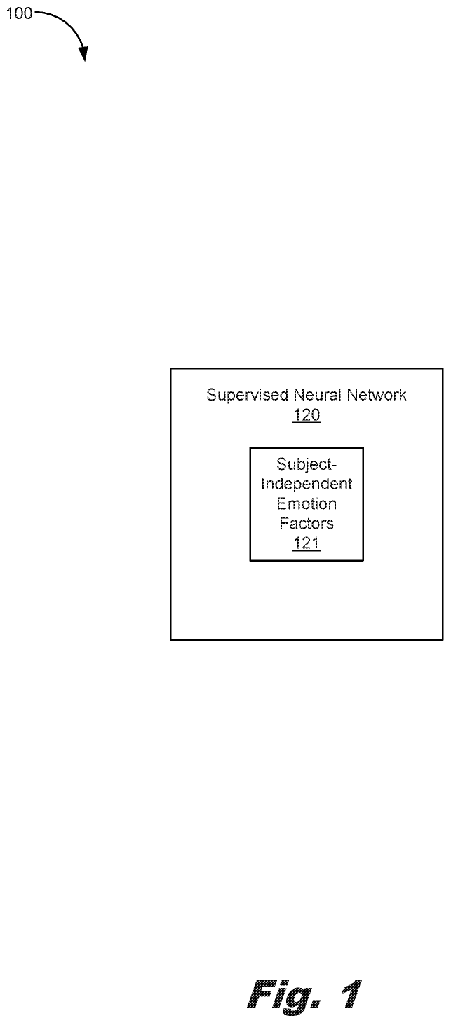

Turning now to the figures, FIG. 1 is a block diagram of a system (100) for classifying physiological data immune to individual variations, according to an example of the principles described herein. The system (100) includes a supervised neural network (120) to classify the physiological data based on the subject-independent emotion factors (121). The subject-independent emotion factors (121) have been isolated within the physiological data from subject-dependent individual factors. By isolating the subject-independent emotion factors (121) from the subject-dependent individual factors or otherwise removing the subject-dependent individual factors from the training of the neural network, the individual's physiological data may be better classified.

In the examples described herein, the physiological data may be physiological data obtained through electroencephalography (EEG). EEG is an electrophysiological monitoring method to record electrical activity of the brain. It may be noninvasive, with the electrodes or other sensors placed along the scalp. EEG measures voltage fluctuations resulting from ionic current within the neurons of the brain. In clinical contexts, EEG refers to the recording of the brain's spontaneous electrical activity over a period of time, as recorded from the multiple electrodes placed on the scalp. However, the present systems and methods may be used in connection with other types of physiological data obtained from other methods such as, for example, electrocardiography (ECG), electromyography (EMG), polygraph, thermometry, pulse oximetry, blood pressure measurement, heart rate monitor, other methods of detecting physiological states of an individual, and combinations thereof.

In one example, a physiological data collection device may be incorporated into an enhanced reality device such that the physiological data is collected during the use of the enhanced reality device. As used in the present specification and in the appended claims, the term “enhanced reality” is meant to be understood broadly as a reality that has been enhanced and presented to a user's senses. An enhanced reality may be presented to a user via, for example, a user interface, a virtual reality (VR) system or device, an augmented reality (AR) system or device, a mixed reality (MR) system or device, and combinations thereof.

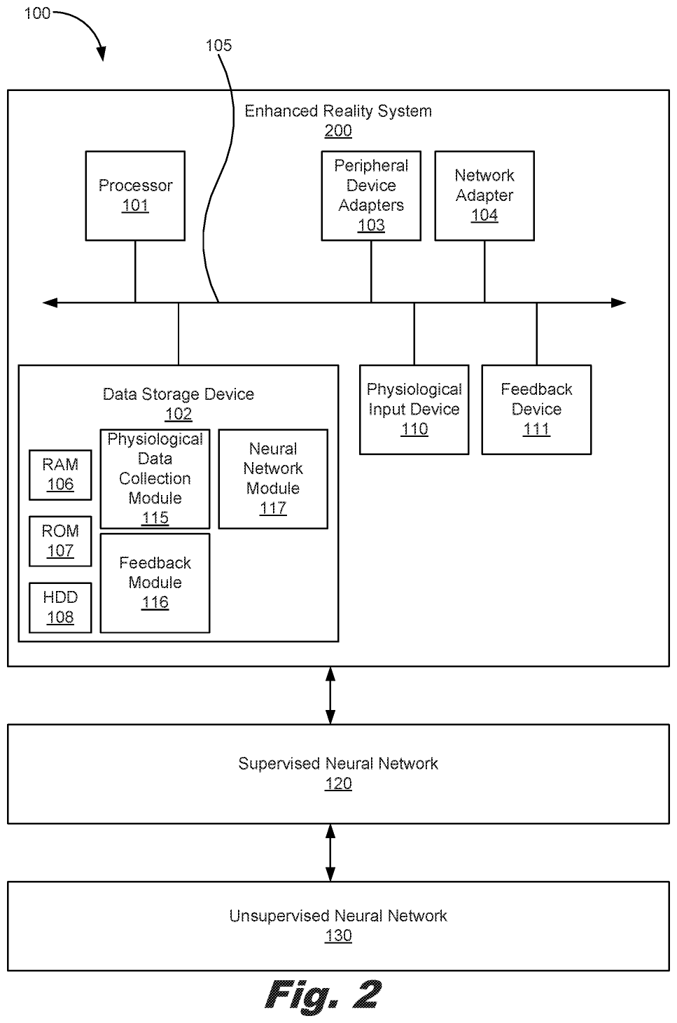

As types of enhanced reality, AR, VR, and MR may involve users interacting with real and/or perceived aspects of an environment in order to manipulate and/or interact with that environment. Interaction by a user in the AR, VR, and/or MR environments may be viewed by others via a display device communicatively coupled to an AR, VR, and/or MR system. AR, VR, and MR systems and devices are used by a user to perceive a visual representation of a VR, AR, and/or MR environments. VR systems and devices implement virtual reality (VR) headsets to generate near real-life, abstract, surreal, and/or realistic images, sounds, and other human discernable sensations that simulate a user's physical presence in a virtual environment presented at the headset. In some examples, the VR system and/or device includes physical spaces and/or multi-projected environments. AR systems and devices may include those systems and devices that implement live direct and/or indirect view of a physical, real-world environment whose elements are augmented by computer-generated sensory input such as sound, video, graphics and/or GPS data. MR systems and devices include the merging of real and virtual worlds to produce new environments and visualizations where physical and digital objects co-exist and interact in real time. For simplicity in description, a virtual reality (VR), augmented reality (AR), and mixed reality (MR) systems and devices are referred to herein as enhanced reality systems and devices. More details regarding the system (100) and its removal of individual variation from emotional representations within physiological signals such as EEG signals is described herein in connection with FIGS. 2 through 5.

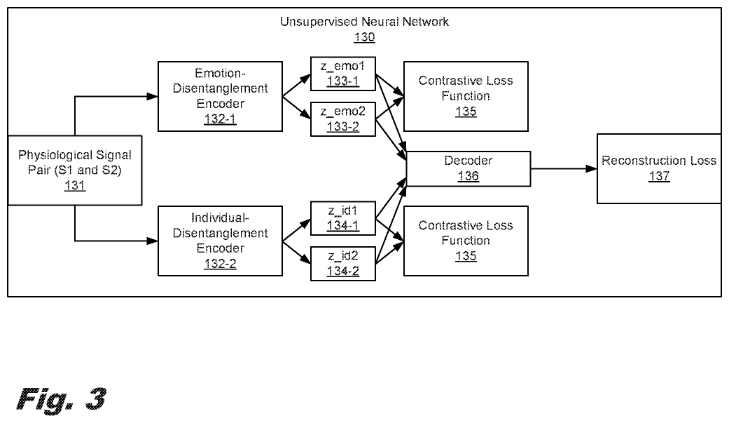

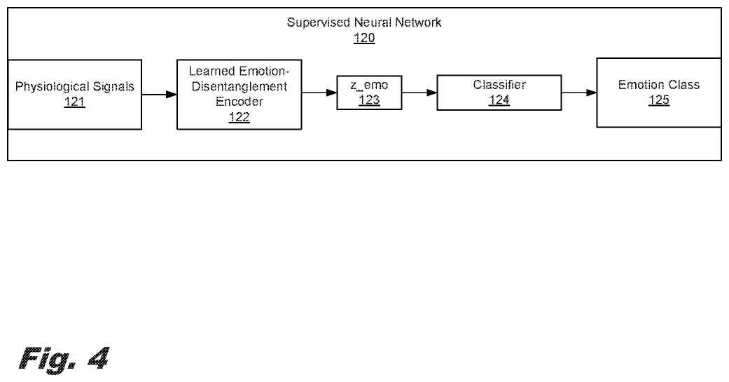

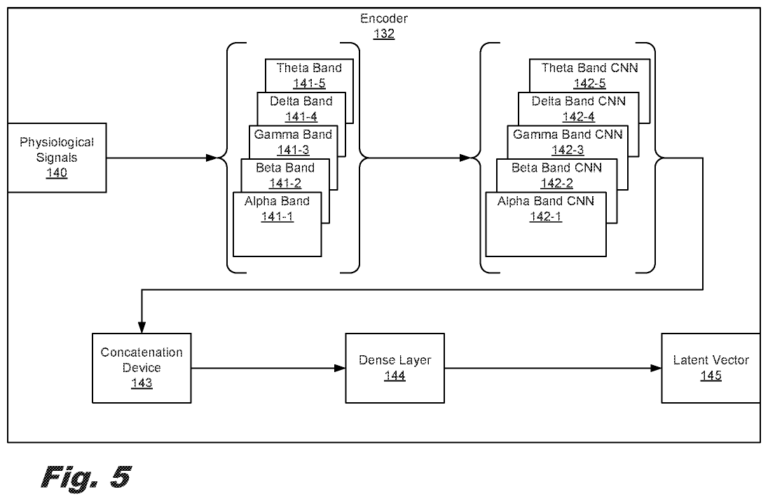

FIG. 2 is a block diagram of a system (100) for classifying physiological data immune to individual variations, according to an example of the principles described herein, and includes an enhanced reality system (200), the supervised neural network (120), and an unsupervised neural network (130). FIG. 3 is a block diagram of the unsupervised neural network (130), according to an example of the principles described herein, and FIG. 4 is a block diagram of the supervised neural network (120), according to an example of the principles described herein. FIG. 5 is a block diagram of an encoder (132), according to an example of the principles described herein. At least one encoder (132) is included within each of the supervised neural network (120), and the unsupervised neural network (130).

The enhanced reality system (200) may be implemented in an electronic device associated with enhanced reality processes such as, for example, a VR hardware, AR hardware, MR hardware, servers, desktop computers, laptop computers, personal digital assistants (PDAs), mobile devices, smartphones, gaming systems, and tablets, among other electronic devices, and combinations thereof.

To achieve its desired functionality, the enhanced reality system (200) includes various hardware components. Among these hardware components may be a processor (101), a data storage device (102), a peripheral device adapter (103), and a network adapter (104), a physiological input device (110), and a feedback device (111). These hardware components may be interconnected and communicatively coupled through the use of a number of busses and/or network connections such as via bus (105).

The processor (101) may include the hardware architecture to retrieve executable code from the data storage device (102) and execute the executable code. The executable code may, when executed by the processor (101), cause the processor (101) to implement at least the functionality of classifying physiological data based on subject-independent emotion factors; separating, with the unsupervised neural network (130), the subject-dependent individual factors of physiological data from subject-independent emotion factors of the physiological data; with an individual-disentanglement encoder (FIG. 3, 132-2), separating the subject-dependent individual factors from a physiological data signal pair (FIG. 3, 131) to create a first individual latent vector (FIG. 3, 134-1) and a second individual latent vector (FIG. 3, 143-2), the separation of the subject-dependent individual factors from the physiological data signal pair creating a learned individual-disentanglement encoder; and with an emotion-disentanglement encoder (FIG. 3, 132-1), separating the subject-independent emotion factors from the physiological data signal pair to create a first emotion latent vector (FIG. 3, 133-1) and a second emotion latent vector (FIG. 3, 133-2), the separation of the subject-independent emotion factors from the physiological data signal pair creating a learned emotion-disentanglement encoder.

Further, the executable code may, when executed by the processor (101), cause the processor (101) to implement at least the functionality of applying a contrastive loss function (FIG. 3, 135) to the first and second individual latent vectors (FIG. 3, 134-1, 134-2), and applying the contrastive loss function (FIG. 3, 135) to the first and second emotion latent vectors (FIG. 3, 131-1, 133-2). The contrastive loss function causes the individual-disentanglement encoder (FIG. 3, 132-2) and the emotion-disentanglement encoder (FIG. 3, 132-1) to map the physiological data signal pair (FIG. 3, 131) as close as possible in individual or emotion latent space if the physiological data signal pair (FIG. 3, 131) are from the same individual or share the same emotion respectively.

Specifically, the contrastive loss function for the emotion-disentanglement encoder (FIG. 3, 132-1) is a distance-based loss analysis that attempts to ensure that semantically similar examples such as the first emotion latent vector (FIG. 3, 133-1) and the second emotion latent vector (FIG. 3, 133-2) are embedded close together. The contrastive loss function for the emotion-disentanglement encoder (FIG. 3, 132-1) is employed to learn the parameters W of a parameterized function G_{W} in such a way that neighbors are pulled together and non-neighbors are pushed apart. Prior knowledge can be used to identify the neighbors for each training data point. The method uses an energy-based model that uses the given neighborhood relationships to learn the mapping function. For a family of functions G, parameterized by W, the objective is to find a value of W that maps a set of high dimensional inputs to the manifold such that the Euclidean distance between points on the manifold, D_{W}(\vec{X}₁,\vec{X}₂)=∥G_{W}(\vec{X}₁)−G_{W}(\vec{X}₂)∥₂ approximates the “semantic similarity” of the inputs in input space, as provided by a set of neighborhood relationships. No assumption is made about the function G_{W} except that it is differentiable with respect to W. Here, X₁ and X₂ are the first emotion latent vector (FIG. 3, 133-1) and the second emotion latent vector (FIG. 3, 133-2), respectively. The contrastive loss function for the emotion-disentanglement encoder (FIG. 3, 132-1) looks at whether the first emotion latent vector (FIG. 3, 133-1) and the second emotion latent vector (FIG. 3, 133-2) are the same and disregards whether the first and second individual latent vectors (FIG. 3, 134-1, 134-2) are the same.

Similarly, the contrastive loss function for the individual-disentanglement encoder (FIG. 3, 132-2) is a distance-based loss analysis that attempts to ensure that semantically similar examples such as the first individual latent vector (FIG. 3, 134-1) and the second individual latent vector (FIG. 3, 143-2) are embedded close together. The same contrastive loss function for the emotion-disentanglement encoder (FIG. 3, 132-1) is also employed for the individual-disentanglement encoder (FIG. 3, 132-2). In this case, however, X₁ and X₂ are the first individual latent vector (FIG. 3, 134-1) and the second individual latent vector (FIG. 3, 143-2), respectively. The contrastive loss function for the individual-disentanglement encoder (FIG. 3, 132-2) looks at whether the first individual latent vector (FIG. 3, 134-1) and the second individual latent vector (FIG. 3, 143-2) are the same and disregards whether the first and second emotion latent vectors (FIG. 3, 133-1, 133-2) are the same.

In one example, the individual-disentanglement encoder (FIG. 3, 132-2) and the emotion-disentanglement encoder (FIG. 3, 132-1) may be variational encoders. A variational encoder may be any device or system that utilizes an encoder, a decoder, and a loss function to approximate inference in a latent Gaussian model where the approximate posterior and model likelihood are parametrized by neural networks.

Further, the executable code may, when executed by the processor (101), cause the processor (101) to implement at least the functionality of, with a decoder (FIG. 3, 136), reconstructing corresponding individual and emotion latent vectors (FIG. 3, 133-1, 133-2, 134-2, 134-2) to output reconstructed data. Classifying the physiological data based on the subject-independent emotion factors may include, for each physiological signal of the physiological data signal pair (FIG. 3, 131), applying the learned emotion-disentanglement encoder (FIG. 4, 122) to classify the subject-independent emotion factors from each physiological data signal (FIG. 3, 131), and classify the subject-independent emotion factors. These and other processes and methods performed by the processor (101) may be according to the methods of the present specification described herein. In the course of executing code, the processor (101) may receive input from and provide output to a number of the remaining hardware units.

The data storage device (102) may store data such as executable program code that is executed by the processor (101) or other processing device. As will be discussed, the data storage device (102) may specifically store computer code representing a number of applications that the processor (101) executes to implement at least the functionality described herein. The data storage device (102) may include various types of memory modules, including volatile and nonvolatile memory. For example, the data storage device (102) of the present example includes Random Access Memory (RAM) (106), Read Only Memory (ROM) (107), and Hard Disk Drive (HDD) memory (108). Many other types of memory may also be utilized, and the present specification contemplates the use of many varying type(s) of memory in the data storage device (102) as may suit a particular application of the principles described herein. In certain examples, different types of memory in the data storage device (102) may be used for different data storage needs. For example, in certain examples the processor (101) may boot from Read Only Memory (ROM) (107), maintain nonvolatile storage in the Hard Disk Drive (HDD) memory (108), and execute program code stored in Random Access Memory (RAM) (106).

The data storage device (102) may include a computer readable medium, a computer readable storage medium, or a non-transitory computer readable medium, among others. For example, the data storage device (102) may be, but not limited to, an electronic, magnetic, optical, electromagnetic, infrared, or semiconductor system, apparatus, or device, or any suitable combination of the foregoing. More specific examples of the computer readable storage medium may include, for example, the following: an electrical connection having a number of wires, a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer readable storage medium may be any tangible medium that can contain, or store computer usable program code for use by or in connection with an instruction execution system, apparatus, or device. In another example, a computer readable storage medium may be any non-transitory medium that can contain, or store a program for use by or in connection with an instruction execution system, apparatus, or device.

The peripheral device adapter (103) and the network adapter (104) in the enhanced reality system (200) enable the processor (101) to interface with various other hardware elements, external and internal to the enhanced reality system (200). For example, the peripheral device adapters (103) may provide an interface to input/output devices, such as, for example, the unsupervised neural network (130), the supervised neural network (120), the physiological input device (110), the feedback device (111), a display device, a mouse, or a keyboard, among other devices. The peripheral device adapters (103) may also provide access to other external devices such as an external storage device, a number of network devices such as, for example, servers, switches, and routers, client devices, other types of computing devices, and combinations thereof. The network adapter (104) may provide an interface to other computing devices within, for example, a network, thereby enabling the transmission of data between the enhanced reality system (200) and other devices located within the network.

The physiological input device (110) may include any device used to collect and distribute physiological data to the unsupervised neural network (130) and the supervised neural network (120). The physiological input device (110) may include any auditory, gustatory, olfactory, visual, somatosensory, or other human perception input device or any sensory modalities thereof. The physiological input device (110) may include, for example, head-mounted displays, biometric sensors, a keyboard, a mouse, gamepads, joysticks, handheld sensing devices, and microphones, among other enhanced reality input devices, and combinations thereof.

The feedback device (111) may include any auditory, gustatory, olfactory, visual, somatosensory, or other human perception feedback device or any sensory modalities thereof. The feedback device (111) may include may include, for example, a display device, a head-mounted display, a speaker, a game pad, a full or partial body haptic suit, an enhanced reality backpack, handled feedback devices, rumble packs, other feedback devices, or combinations thereof.

The enhanced reality system (200) further includes a number of modules used in the implementation of the methods and processes described herein. The various modules within the enhanced reality system (200) include executable program code that may be executed separately. In this example, the various modules may be stored as separate computer program products. In another example, the various modules within the enhanced reality system (200) may be combined within a number of computer program products; each computer program product including a number of the modules.

The enhanced reality system (200) may include a physiological data collection module (115) to, when executed by the processor (101), collect physiological data via the physiological input device (110). In one example, the physiological data collection module (115) may collect physiological data from an array of biometric sensors attached to an individual. A number of physiological signals may be obtained from the biometric sensors, and these physiological signals may be grouped to form the physiological signal pairs (S1 and S2) (FIG. 2, 131) used as input to the unsupervised neural network (130).

The enhanced reality system (200) may include a feedback module (116) to, when executed by the processor (101), provide feedback to an individual based on the classified physiological data. The outcome of the classification of the physiological data may be, for example, a classification of an emotion. Once the enhanced reality system (200) obtains classified emotions, the enhanced reality system (200) may use the feedback module (116) and the feedback device (111) to provide appropriate feedback to the user based on the emotion that was classified. Although emotions are described herein as being the example of the physiological data that is classified, any type of physiological data may be classified using the systems and methods described herein.

The enhanced reality system (200) may include a neural network module (116) to, when executed by the processor (101), may interact with the supervised neural network (120) and the unsupervised neural network (130) to classify physiological data obtained via the physiological data collection module (115) and provide feedback based on the output of the supervised neural network (120) and the unsupervised neural network (130) via the execution of the feedback module (116).

Turning to FIG. 3 specifically, FIG. 3 is a block diagram of an unsupervised neural network (130), according to an example of the principles described herein. The unsupervised neural network (130) provides autoencoders (132-1, 132-2) with contrastive regularization to separate original data into subject-dependent individual factors and subject-independent emotion factors. The unsupervised neural network (130) provides a semi-supervised pre-train process and the supervised neural network (120) provides a supervised fine-tuning process and a classification process.

In the pre-train process provided by the unsupervised neural network (130), a number of physiological signal pairs (131) comprising a first signal (S1) and a second signal (S2) are input into the unsupervised neural network (130) form the execution of the physiological data collection module (115) and the output of the physiological input device (110) of the enhanced reality system (200). The physiological signal pairs (131) include both subject-independent physiological factors (e.g., subject-independent emotional factors) and subject-dependent individual factors. The subject-independent emotional factors in this example are what the present systems and methods seek to classify in isolation from the subject-dependent individual factors. The subject-dependent individual factors within the physiological signal pairs (131) may bias a subsequent classification of the physiological data, and, therefore, are not desirable as a component within the classification processes described herein. Training in pairs provides for the contrastive regularization provided by the unsupervised neural network (130). If pairs of signals are not used, and one signal is used, then a proper and effective weight distribution cannot be determined in the latent space. The two signals within the pair are compared in order to train the unsupervised neural network.

Thus, the physiological signal pairs (131) are isolated into subject-independent physiological factors and subject-dependent individual factors using two encoders: the emotion variations encoder (132-1) and the individual variations encoder (132-2). Each of these encoders produce latent vectors. The emotion variations encoder (132-1) produces a first emotion latent vector z_emo1 (133-1) corresponding to signal S1 of the physiological data signal pair (131) and a second emotion latent vector z_emo2 (133-2) corresponding to signal S2 of the physiological data signal pair (131). Similarly, the individual variations encoder (132-2) produces a first individual latent vector z_id1 (134-1) corresponding to signal S1 of the physiological data signal pair (131) and a second individual latent vector z_id2 (134-2) corresponding to signal S2 of the physiological data signal pair (131).

In order to describe the function of the emotion variations encoder (132-1) and the individual variations encoder (132-2) as well as the learned emotion-disentanglement encoder (FIG. 4, 122), a description of the function of an encoder will be described in connection with FIG. 5. FIG. 5 is a block diagram of an encoder (132), according to an example of the principles described herein. An encoder (122, 132) includes a number of bands (141-1, 141-2, 141-3, 141-4, 141-5, collectively referred to herein as 141) to receive as input physiological signals (140) which, in this example, are EEG signals. The physiological signals (140) are subdivided into bandwidths such as an alpha band (141-1), a beta band (141-2), a gamma band (141-3), a delta band (141-4), and a theta band (141-5), to signify a number of the EEG bandwidths used in clinical practice. Each band (141) may contain a feature that the encoder (132) can identify.

More or fewer bands (141) may be used on connection with the input physiological signals (140) input into the encoder (132). A corresponding number of parallel convolutional neural network (CNN) layers (142-1, 142-2, 142-3, 142-4, 142-5, collectively referred to herein as 142) may be applied to all five bands (141) separately. Each of the individual applications of the parallel CNN layers (142) may include non-shared weights where different weights are applied to the individual layers (142) of the CNN. A CNN is, in machine learning, a class of deep, feed-forward artificial neural networks, that may be applied to analyzing visual imagery. CNNs use a variation of multilayer perceptrons based on their weights architecture and translation invariance characteristics. The parallel CNN layers (142) each apply their respective weights to the bands (141). An architecture of the encoders is described herein in connection with Table 1:

TABLE 1
Architecture for encoders

| Operation | Input Shape | Kernel Size | Output Shape |
|---|---|---|---|
| Inputs | (k, 5, 185, 62) | | (k, 5, 185, 62) |
| Split band | (k, 5, 185, 62) | | 5*(k, 185, 62) |
| Each band | 5*(k, 185, 62) | | (k, 185, 62) |
| Conv | (k, 185, 62) | (7, 62, 128) | (k, 93, 128) |
| Conv | (k, 93, 128) | (3, 128, 256) | (k, 47, 256) |
| Conv | (k, 47, 256) | (3, 256, 256) | (k, 24, 256) |
| FC | (k, 24, 256) | (24*256, 50) | (k, 50) |
| Concat | 5*(k, 50) | | (k, 250) |
| FC | (k, 250) | (250, 1000) | (k, 1000) |
| FC | (k, 1000) | (1000, 100) | (k, 100) |

In Table 1, the layers are listed under the “operations” column and are those hidden layers in the neural network on which the neural network operates. Here, “Conv” denotes convolutional layer with stride equaling 2. “FC” denotes a fully-connected layer. As to the input shape of the input signals, the k value indicates the batch size. In other words, k pairs of signals are input to the neural network in each iteration described herein. The number 5 as the second entry in the input shape of the inputs column indicates the number of bands processed by the encoder (132). Here there are 5 bands created from the EEG signal and these 5 bands may be created based on a division between frequencies of the signal. The number 185 as the third entry in the input shape of the inputs column indicates a signal length. In this example, the signal has 185 samples. The number 62 as the fourth entry in the input shape of the inputs column indicates the number of channels from which the input comes. In one example, there may be multiple sensors located in an array of sensors within the EEG system that is used to collect the physiological data, and the channel indicates the number of sensors in the array form which the signals are coming from. The kernel size of a convolutional kernel is the window length of the kernel. In the fourth row of entries under “onv” the kernel size is 7. Further; the input channel is 62 and output channel is 128 meaning that there are 128 output feature maps once the convolutional process is completed. The output shape of the architecture presented in Table 1 follows the same pattern as presented in the input shape. The architecture depicted in Table 1 is merely an example, and the CNN may have any architecture as may prove effective in a given scenario.

Turning again to FIG. 5, the encoder (132) fuses the parallel CNN layers (142) through concatenation using a concatenation device (143), and the fused, concatenated layers are provided as input to latter layers as a dense layer (144). The dense layer (144) forms a latent vector (145). This latent vector (145) is analogous to the first emotion latent vector (FIG. 3, 133-1), second emotion latent vector (FIG. 3, 133-2), the first individual latent vector (FIG. 3, 134-1), and the second individual latent vector (FIG. 3, 143-2) as formed by their respective encoders (132-1, 132-2). The process performed by the encoder (132) is similar to an inception network. However, instead of stacking all channels at the beginning, processing certain tasks separately and fusing them later in the processing may be more effective for learning critical information based on certain domain knowledge including, for example, the five EEG bands (141).

Similarly, in the decoder (FIG. 3, 136), whose architecture is provided in Table 2, once the emotion vector (133-1, 133-2) and individual vector (134-1, 134-2) are input therein, the decoder (136) may concatenate the vectors (133-1, 133-2, 134-1, 134-2) together and copy it five times for input to the reconstruction loss (137) for each frequency band.

TABLE 2
Architecture for Decoders. “Upconv” is transposed-convolutional layer
with stride = 2. “FC” is fully-connected layer.

| Operation | Input Shape | Kernel Size | Output Shape |
|---|---|---|---|
| Concat | 2*(k, 100) | | (k, 200) |
| Split band | (k, 200) | | 5*(k, 200) |
| Each band | 5*(k, 200) | | (k, 512) |
| FC | (k, 200) | (200, 11*512) | (k, 11, 512) |
| Upconv | (k, 11, 512) | (3, 512, 256) | (k, 23, 256) |
| Upconv | (k, 23, 256) | (3, 256, 256) | (k, 46, 256) |
| Upconv | (k, 46, 256) | (3, 256, 128) | (k, 92, 128) |
| Upconv | (k, 92, 128) | (3, 128, 62) | (k, 185, 62) |
| Concat | 5*(k, 185, 62) | | (k, 5, 185, 62) |

The output of the emotion variations encoder (132-1) and the individual variations encoder (132-2) is the emotion latent vectors z_emo1 (133-1) and z_emo2 (133-2) and the individual latent vectors z_id1 (134-1) and z_id2 (134-2). These latent vectors (133-1, 133-2, 134-1, 134-2) are then input to a contrastive loss function (135). A contrastive loss function (135) treats data sets in such a way that neighbors are pulled together and non-neighbors are pushed apart. Thus, the contrastive loss function (135) is used to constrain the learning process. Each pair of data samples has two corresponding labels “same emotion” and “same individual” to represent if this pair have same emotion label and same individual label respectively. If the physiological signal pair S1 and S2 share the same emotion or are from the same person, it is desirable that the encoders (132-1, 132-2) be able to map them into the corresponding latent space as close as possible. In contrast, if the physiological signal pair S1 and S2 do not share the same emotion or are not from the same person, it is desirable that the encoders (132-1, 132-2) map them within the corresponding latent space at a relatively larger margin such as a threshold margin away from one another. This margin or threshold is set by the contrastive loss function (135). The contrastive loss function (135) causes the emotion variations encoder (132-1) and the individual variations encoder (132-2) to map the physiological data signal pair (131) as close as possible in latent space if the physiological data signal pair (131) are from the same individual or share the same emotion respectively. The value output by the contrastive loss function (135) is added to the total loss of the unsupervised neural network (130) as part of the reconstruction loss (137).

The unsupervised neural network (130) may then use a decoder (136) to concatenate the vectors (133-1, 133-2, 134-1, 134-2) together and copy it five times for input to the reconstruction loss device (137) for each frequency band. The decoder (136) will combine the first emotion vector (133-1) with the first individual vector (134-1), and will combine the second emotion vector (133-3) with the second individual vector (134-2) and create two separate inputs from these two combinations. The decoder (136) may then perform a transposed convolution process to attempt to reconstruct the original signals S1 and S2. The value at the reconstruction loss (137) may be small in cases where the original signals S1 and S2 are similar to the value at the decoder (136).

The system (100) includes the unsupervised neural network (130) in order to identify features of the signals S1 and S2 that may be used in the supervised neural network (120), and, specifically, the classifier (FIG. 4, 124) of the supervised neural network (120). The input of the supervised neural network (120) is a single physiological data signal. The single signal is then analyzed by a now learned emotion-disentanglement encoder (122) to recognize the emotion latent vector of the signal and not the individual latent vector. In the examples described herein, the learned emotion-disentanglement encoder (122) may include the training data obtained by the emotion-disentanglement encoder (FIG. 3, 132-1), may be an embodiment of the emotion-disentanglement encoder (FIG. 3, 132-1). or may be the emotion-disentanglement encoder (FIG. 3, 132-1) itself. The output of the learned emotion-disentanglement encoder (122) is emotion latent vector z_emo (123); a feature learned in the unsupervised neural network (130). The emotion latent vector z_emo (123) is then classified by the classifier (124). The classifier (124) may be any type of neural network used to classify latent vectors of physiological data. In one example, the classifier (124) classifies the emotion latent vector z_emo (123) into an emotion class (125) such as a positive, neutral, or negative emotion. However, in another example, additional categories of classifications may be included.

Once the emotion latent vector z_emo (123) has been classified by the classifier (124) into an emotion class (125), this classification may be used to enhance a user's experience with the enhanced reality system (200). For example, where the physiological data input to the unsupervised neural network (130) and the supervised neural network (120) is EEG data, the classified emotion latent vector may be used as feedback to an enhanced reality shopping instance where the user is using the enhanced reality system (200) to perform online shopping. In this example, the classified emotion latent vector may inform the system (100) whether the user has a positive, neutral, or negative opinion of a product, and may provide additional products based on the emotion detected by the system (100).

In another example, an enhanced reality training simulation such as a firefighting training simulation using the enhanced reality system (200) may be able to use the unsupervised neural network (130) and the supervised neural network (120) to understand how the user in the simulation is affected by the simulation. Feedback may be provided to the user after the simulation is done to show to the user when he or she had positive, neutral, and negative emotions during the duration of the simulation.

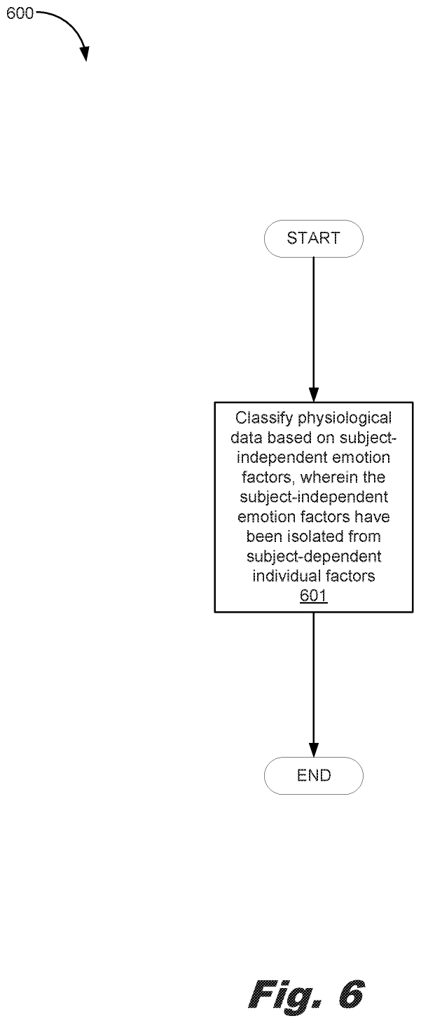

Having described the function of the various elements of the system (100) including the enhanced reality system (200), the unsupervised neural network (130) and the supervised neural network (120), and each of their various elements, one purpose of the system (100) is to disentangle the subject-independent physiological factors from the subject-dependent individual factors or otherwise remove the subject-dependent individual factors from the training of the neural network. This provides a more consistent and non-biased classification of the physiological data. Any type of physiological data may be processed according to the systems and methods described herein. More details are provided herein in connection with the methods of FIGS. 6 through 9.

FIG. 6 is a flowchart showing a method (600) of removing individual variation from emotional representations, according to an example of the principles described herein. The method (600) may include classifying (block 601) physiological data based on subject-independent emotion factors. The subject-independent emotion factors are analogous to the first emotion latent vector (FIG. 3, 133-1) and a second emotion latent vector (FIG. 3, 133-2). The subject-independent emotion factors have been isolated from subject-dependent individual factors. The subject-dependent individual factors are analogous to the first individual latent vector (FIG. 3, 134-1) and a second individual latent vector (FIG. 3, 143-2) identified and isolated by the unsupervised neural network (130) and disregarded by the supervised neural network (120).

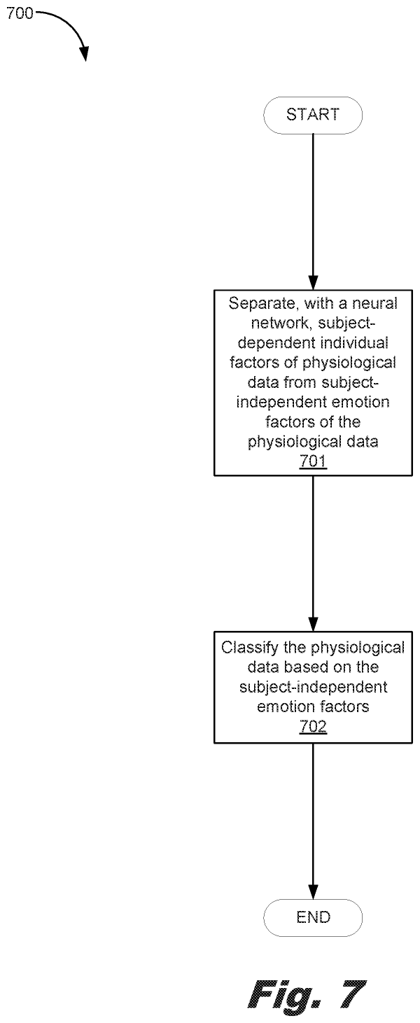

FIG. 7 is a flowchart showing a method (700) of removing individual variation from emotional representations, according to an example of the principles described herein. The method (700) may include separating (block 701), with an unsupervised neural network (130), subject-dependent individual factors of physiological data from subject-independent emotion factors of the physiological data. The physiological data may be classified (block 702) by the supervised neural network (120) based on the subject-independent emotion factors using a learned emotion-disentanglement encoder (122) of the supervised neural network (120).

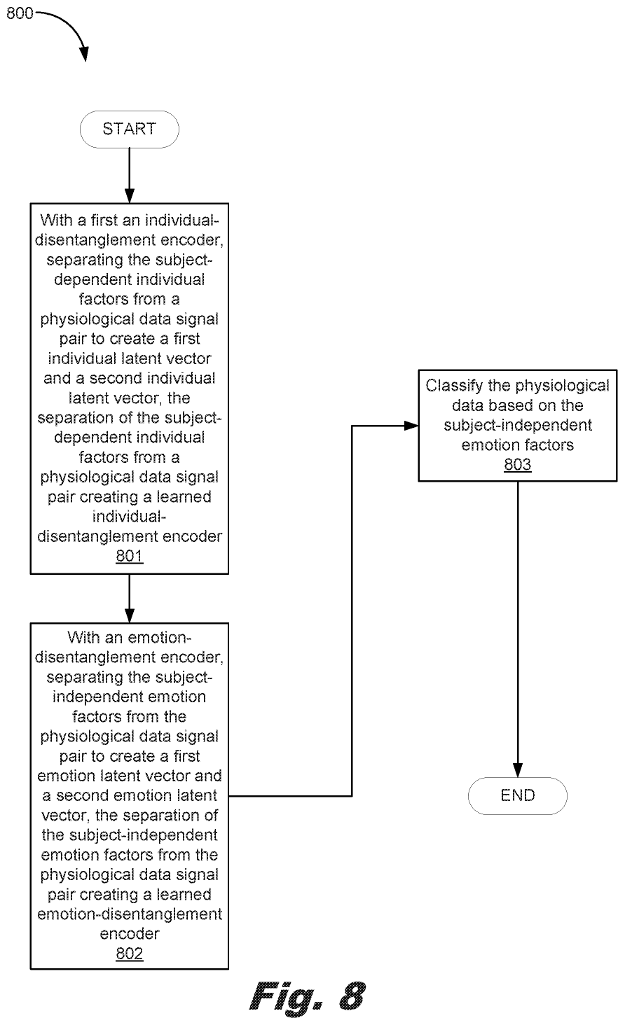

FIG. 8 is a flowchart showing a method (800) of removing individual variation from emotional representations, according to an example of the principles described herein. The method (800) may include, with an individual-disentanglement encoder (132-2), separating (block 801) the subject-dependent individual factors from a physiological data signal pair (131) to create a first individual latent vector (134-1) and a second individual latent vector (134-2). The separation of the subject-dependent individual factors from the physiological data signal pair creates the learned individual-disentanglement encoder (132-2).

Further, the method (800) may include, with an emotion-disentanglement encoder (132-1), separating (block 802) the subject-independent emotion factors from the physiological data signal pair to create a first emotion latent vector (133-1) and a second emotion latent vector (133-2). The separation of the subject-independent emotion factors from the physiological data signal pair creates the learned emotion-disentanglement encoder (132-1). The physiological data may be classified (block 803) by the supervised neural network (120) based on the subject-independent emotion factors using a learned emotion-disentanglement encoder (122) of the supervised neural network (120). The learned emotion-disentanglement encoder (122) was trained based on the manner in which the individual factors of the physiological data were isolated or removed from the subject-independent emotion factors of the physiological data as performed by the emotion variations encoder (132-1) and the individual variations encoder (132-2). Thus, in the examples described herein, the learned emotion-disentanglement encoder (122) may include the training data obtained by the emotion-disentanglement encoder (FIG. 3, 132-1), may be an embodiment of the emotion-disentanglement encoder (FIG. 3, 132-1). or may be the emotion-disentanglement encoder (FIG. 3, 132-1) itself.

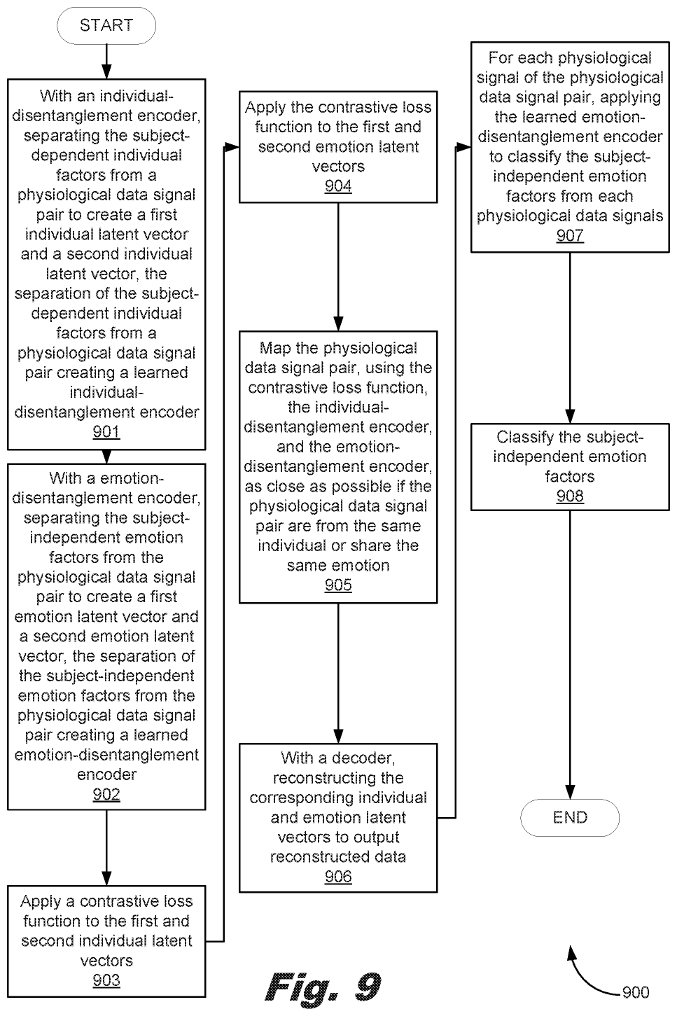

FIG. 9 is a flowchart showing a method (900) of removing individual variation from emotional representations, according to an example of the principles described herein. The method (900) may include, with an individual-disentanglement encoder (132-2), separating (block 901) the subject-dependent individual factors from a physiological data signal pair (131) to create a first individual latent vector (134-1) and a second individual latent vector (134-2). The separation of the subject-dependent individual factors from the physiological data signal pair creates the learned individual-disentanglement encoder (132-2).

Further, the method (900) may include, with an emotion-disentanglement encoder (132-1), separating (block 902) the subject-independent emotion factors from the physiological data signal pair to create a first emotion latent vector (133-1) and a second emotion latent vector (133-2). The separation of the subject-independent emotion factors from the physiological data signal pair creates the learned emotion-disentanglement encoder (132-1).

A contrastive loss function (135) may be applied (bock 903) to the first and second individual latent vectors (134-1, 134-2). Similarly, the contrastive loss function (135) may be applied (bock 904) to the first and second emotion latent vectors (133-1, 133-2). The method (900) may also include mapping (block 905) the physiological data signal pair (131), using the contrastive loss function (135), the individual-disentanglement encoder (132-2), and the emotion-disentanglement encoder (132-1), as close as possible if the physiological data signal pair are from the same individual or share the same emotion.

The method (900) may also include, with a decoder (136), reconstructing (block 906) the corresponding individual and emotion latent vectors to output reconstructed data (137). For each physiological signal, S1 and S2, of the physiological data signal pair (131), the learned emotion-disentanglement encoder (132-1) may be applied (block 907) to classify the subject-independent emotion factors from each of the physiological data signals, S1 and S2. The subject-independent emotion factors may be classified (bock 908) using the learned emotion-disentanglement encoder (122) of the supervised neural network (120).

Aspects of the present systems and methods are described herein with reference to flowchart illustrations and/or block diagrams of methods, apparatus (systems) and computer program products according to examples of the principles described herein. Each block of the flowchart illustrations and block diagrams, and combinations of blocks in the flowchart illustrations and block diagrams, may be implemented by computer usable program code. The computer usable program code may be provided to a processor of a general purpose computer, special purpose computer, or other programmable data processing apparatus to produce a machine, such that the computer usable program code, when executed via, for example, the processor (101) of the enhanced reality system (200) or other programmable data processing apparatus, implement the functions or acts specified in the flowchart and/or block diagram block or blocks. In one example, the computer usable program code may be embodied within a computer readable storage medium; the computer readable storage medium being part of the computer program product. In one example, the computer readable storage medium is a non-transitory computer readable medium.

The specification and figures describe a method of removing individual variation from emotional representations. The method may include classifying physiological data based on subject-independent emotion factors. The subject-independent emotion factors are isolated from subject-dependent individual factors. Further, the specification and figures describe a non-transitory computer readable medium includes computer usable program code embodied therewith. The computer usable program code, when executed by the processor classifies, with a first neural network, the physiological data based on subject-independent emotion factors from the trained first neural network. The subject-independent emotion factors have been isolated within the physiological data from subject-dependent individual factors.

The systems and methods described herein increases the manner in which the system (100) and the enhanced reality system (200) functions by assisting in the manner in which physiological data is identified and classified and outputting of instructions based on that more efficient, precise and accurate classification of the physiological data. With the anonymized signal produced by isolating the subject-dependent individual factors from the subject-independent emotion factors, the emotion recognition system achieves higher recognition accuracy and lower variability across individuals.

The preceding description has been presented to illustrate and describe examples of the principles described. This description is not intended to be exhaustive or to limit these principles to any precise form disclosed. Many modifications and variations are possible in light of the above teaching.

What is claimed is:

1. A method of removing individual variation from emotional representations, comprising:
separating, with at least one encoder of a neural network, subject-dependent individual factors of physiological data from subject-independent emotion factors of the physiological data;
classifying the physiological data based on subject-independent emotion factors,
wherein the subject-independent emotion factors have been isolated from subject-dependent individual factors.

2. The method of claim 1, wherein separating the subject-dependent individual factors of physiological data from subject-independent emotion factors of the physiological data comprises:
with an individual-disentanglement encoder, separating the subject-dependent individual factors from a physiological data signal pair to create a first individual latent vector and a second individual latent vector, the separation of the subject-dependent individual factors from the physiological data signal pair creating a learned individual-disentanglement encoder; and
with an emotion-disentanglement encoder, separating the subject-independent emotion factors from the physiological data signal pair to create a first emotion latent vector and a second emotion latent vector, the separation of the subject-independent emotion factors from the physiological data signal pair creating a learned emotion-disentanglement encoder.

3. The method of claim 2, comprising:
applying a contrastive loss function to the first and second individual latent vectors; and
applying the contrastive loss function to the first and second emotion latent vectors.

4. The method of claim 3, wherein the contrastive loss function causes the individual-disentanglement encoder and the emotion-disentanglement encoder to map the physiological data signal pair as close as possible if the physiological data signal pair are from the same individual or share the same emotion.

5. The method of claim 2, comprising, with a decoder, reconstructing corresponding individual and emotion latent vectors to output reconstructed data.

6. The method of claim 1, wherein classifying the physiological data based on the subject-independent emotion factors comprises:
for each physiological signal of a physiological data signal pair, applying a learned emotion-disentanglement encoder to classify the subject-independent emotion factors from each physiological data signal; and
classifying the subject-independent emotion factors.

7. A non-transitory computer readable medium comprising computer usable program code embodied therewith, the computer usable program code to, when executed by the processor:
with a first neural network, classify the physiological data based on subject-independent emotion factors from the trained first neural network,
wherein the subject-independent emotion factors have been isolated within the physiological data from subject-dependent individual factors.

8. The computer readable medium of claim 7, comprising computer usable program code to, when executed by the processor:
train a second neural network to isolate subject-dependent individual factors of physiological data from subject-independent emotion factors of the physiological data; and
with an individual-disentanglement encoder of the first neural network, separate the subject-dependent individual factors from a physiological data signal pair to create a first individual latent vector and a second individual latent vector, the separation of the subject-dependent individual factors from a physiological data signal pair creating a learned individual-disentanglement encoder; and
with an emotion-disentanglement encoder, separate the subject-independent emotion factors from the physiological data signal pair to create a first emotion latent vector and a second emotion latent vector, the separation of the subject-independent emotion factors from the physiological data signal pair creating a learned emotion-disentanglement encoder.

9. The computer readable medium of claim 8, comprising computer usable program code to, when executed by the processor:
apply a contrastive loss function to the first and second individual latent vectors;
apply the contrastive loss function to the first and second emotion latent vectors; and
with a decoder, reconstruct the corresponding individual latent vectors and emotion latent vectors to output reconstructed data, wherein the contrastive loss function causes the individual-disentanglement encoder and the emotion-disentanglement encoder to map the physiological data signal pair as close as possible if the physiological data signal pair are from the same individual or share the same emotion.

10. The computer readable medium of claim 7, wherein classifying the physiological data based on the subject-independent emotion factors comprises:
for each physiological signal, applying the learned emotion-disentanglement encoder to classify the subject-independent emotion factors from each physiological data signal; and
classify the subject-independent emotion factors.

11. The computer readable medium of claim 8, comprising computer usable program code to, when executed by the processor:
for each of the individual-disentanglement encoder and the emotion-disentanglement encoder:
apply a plurality of convolutional layers of a parallel convolutional neural network (CNN) to a corresponding number of bands of the physiological data; and
concatenate features learned from channels of the parallel CNN to produce the first individual latent vector, the second individual latent vector, the first emotion latent vector, and the second emotion latent vector, respectively.

12. The computer readable medium of claim 11, wherein weighting factors applied to each of the of the of convolutional layers are not the same.

13. A system for classifying physiological data immune to individual variations, comprising:
a physiological input device to collect physiological data;
a supervised neural network to classify the physiological data based on subject-independent emotion factors,
wherein the subject-independent emotion factors have been isolated within the physiological data from subject-dependent individual factors.

14. The system of claim 13, comprising:
an unsupervised neural network to isolate the subject-dependent individual factors of physiological data from the subject-independent emotion factors of the physiological data, comprising:
an individual-disentanglement encoder to separate the subject-dependent individual factors from the physiological data to create a first individual latent vector and a second individual latent vector;
second variational encoder to separate the subject-independent emotion factors from the physiological data to create a first emotion latent vector and a second emotion latent vector;
a first contrastive loss module to apply a contrastive loss function to the first and second individual latent vectors;
a second contrastive loss module to apply the contrastive loss function to the first and second emotion latent vectors; and
a decoder to reconstruct the corresponding individual latent vector and emotion latent vector to output reconstructed data,
wherein the second variational encoder is applied to the supervised neural network as a machine learning process, the second variational encoder being trained based on the creation of the first emotion latent vector and the second emotion latent vector.

15. The system of claim 13, wherein:
the system is an enhanced reality system, and
the physiological data is obtained from a peripheral augmented reality input device.
